(19) United States
(12) Patent Application Publication
(10) Pub. No.: US 2006/0064020 A1
(43) Pub. Date: Mar. 23, 2006

(54) Clinic dashboard monitor

(75) Inventors: John E. Burnes, Andover, MN (US); Luc R. Mongeon, Minneapolis, MN (US)

Correspondence Address: MEDTRONIC, INC., 710 MEDTRONIC PARK, MINNEAPOLIS, MN, 55432-9924, US

(73) Assignee: Medtronic, Inc.

(21) Appl. No.: 10/944,982

(22) Filed: Sep. 20, 2004

(51) Int. Cl. A61B 5/02

(52) U.S. Cl. 600/481; 128/903

(57) ABSTRACT

The present invention provides a remote patient monitoring system including a graphical user interface (GUI) that displays a summary table of categorized parameter values for multiple patients simultaneously. The remote patient monitoring system further includes a central database for receiving data from remote medical devices via a communications network and a processor for parameterizing and categorizing summary data to be displayed by the GUI. The displayed summary parameter values are formatted according to category to allow a parameter value category to be visually recognized. In one embodiment, parameter values are categorized according to a need for clinical attention such that a clinician may view a summary table of categorized parameter values and recognize which patients may require clinical attention as indicated by the formatted parameter values.

### FIELD OF THE INVENTION

The present invention relates generally to remote patient monitoring systems.

### BACKGROUND OF THE INVENTION

Implantable medical devices (IMDs) such as cardiac pacemakers, cardioverters and defibrillators (ICDs), hemodynamic monitors, and drug delivery devices, are being offered with increasing capacity for storing physiological and device performance data. The use of home monitoring instrumentation and equipment, e.g., to measure weight, systemic blood pressure, symptoms, etc., is gaining popularity for managing patients with chronic illnesses. Physiological sensors for monitoring various patient conditions such as heart rhythm, blood pressure, respiration, patient activity level, heart wall motion, and blood chemistry may operate in conjunction with an IMD and home based instrumentation for acquiring continuous or periodic physiological data for processing and/or storage by the IMD or for clinical management. Such data may be used by the IMD in automated therapy delivery or by a clinician in diagnosing or monitoring a patient condition and in his or her therapy management.

Typically, a pacemaker or ICD patient is seen once every three to six months, as long as they are not experiencing adverse symptoms, in order to collect and review data stored by the device. In many cases, the patient may be doing fine and the IMD is operating correctly therefore no clinical action is required. The development of remote patient monitoring systems that allow IMD data to be transferred from the patient's IMD to a home monitor and from the home monitor to a central database (or from the IMD directly to the central database) allows a clinician to reduce the required number of scheduled office visits when a patient is doing well and still maintain an up-to-date review of device performance and stored physiological data.

With the unprecedented amount of clinical data available from IMDs, and data collected by the in-home external medical devices, however, a clinician has a mounting task in tracking and analyzing each patient's data in order to recognize events or conditions of clinical importance or concern. The clinician must also recognize if a remote monitoring session for a particular patient is overdue. Remote monitoring of patients potentially enables a clinician to increase the number of patients he or she is treating by reducing the number of unnecessary office visits. However, having more patients monitored remotely increases the amount of data the clinician is responsible for tracking. Therefore, as data storage capabilities of medical devices increases and remote monitoring systems become more widely used, a need arises for remote monitoring systems and data management methods that relieve the data analysis burden placed on the clinician while enabling the clinician to maintain thorough patient care.

### BRIEF SUMMARY OF THE INVENTION

The present invention provides a remote patient monitoring system including a graphical user interface (GUI) that displays summary data for multiple patients simultaneously. The summary data provides a clinician or other user an overview of the status of multiple patients and highlights patients or conditions that may warrant clinical attention. The remote patient monitoring system further includes a central database for receiving data from remote medical devices (both implanted and external) via a communications network and a processor for generating summary data to be displayed on the GUI. The central database includes multiple patient data records for storing data received from remote medical devices, which may include more than one type or model of medical device. The GUI displays summary data received from the processor for multiple patients simultaneously allowing a clinician to easily scan summary data for a given patient group.

Patient data to be displayed simultaneously may be grouped according to treating clinician and the type of medical device from which the data was received. Patient data may alternatively be grouped according to primary diagnosis, time since last remote monitoring session, indicator of patient status, indicator of device status, or other grouping criteria. Summary data for a patient group may be listed in a tabular format wherein multiple parameter values are listed. Parameter values may include, for example, time since or the date of the last remote monitoring session, device type, patient weight, reported patient symptoms, physiological conditions monitored by the medical device(s), a medical device status indicator, and an overall indicator of the patient status. Any parameter value that is determined to potentially warrant clinical attention may be highlighted or otherwise displayed in a conspicuous manner in the summary display.

Summary parameter values are categorized and displayed according to a symbolic or stylistic scheme that allows the user to quickly recognize the category of any given parameter value. Parameter values are categorized, for example, according to predetermined threshold levels or ranges, the relationship between two or more parameters, or other mathematical or statistical relationship. Parameter value categories are used to indicate a level of clinical severity or need for medical attention. In some embodiments, parameter values are displayed in alphanumeric values, which may be formatted in color-coded text or according to other style schemes, which allow a parameter value category to be recognized. In other embodiments, categorized parameters are displayed as a symbol, icon or index, which may additionally be color-coded or otherwise formatted to enhance quick recognition of the parameter category.

In one embodiment, categorized parameter values are displayed by a red, yellow, or green symbol where a red symbol indicates the parameter value falls in a range that indicates a need for clinical attention, a yellow symbol indicates the parameter value is approaching or near a value that indicates a need for clinical attention, and a green symbol indicates the parameter value is in a normal range that does not warrant clinical attention. A user may scan a display of multiple patient records showing multiple categorized parameter values for each patient and quickly recognize which patients may need further clinical review or attention.

In yet another embodiment, a user may select a patient record or a particular cell within a patient record shown on the GUI summary display to view additional data for that patient. For example, the user may select a parameter value categorized as “needs attention,” and the GUI will display a view of more detailed data relating to the particular parameter value. In this way, the user may review data stored by the central database in greater detail and review data used by the processor in determining the categorized parameter value.

### BRIEF DESCRIPTION OF THE DRAWINGS

FIG. 1 is a schematic diagram illustrating data transmission to a centralized database from a deployed medical device for remote monitoring of a patient.

FIG. 2 schematically illustrates selected components of an implantable medical device.

FIG. 3 is a simplified block diagram of selected functional components typically included in an external medical device having bidirectional communication with an implantable medical device.

FIG. 4 is a schematic diagram illustrating a remote monitoring system configured to acquire data from multiple deployed medical devices and display summary data for multiple patients simultaneously in accordance with the present invention.

FIG. 5 is a flow chart summarizing one method for obtaining and displaying patient summary data.

FIG. 6 is a flow chart summarizing steps included in one method for categorizing and displaying summary data for patients being monitored remotely.

FIG. 7 illustrates one embodiment of an interactive display of summary data for multiple patients being monitored remotely.

FIG. 8 is an illustration of a user-interactive display listing data relating to a device status indicator.

FIG. 9 illustrates an interactive display listing detailed patient data.

### DETAILED DESCRIPTION OF THE PREFERRED EMBODIMENTS

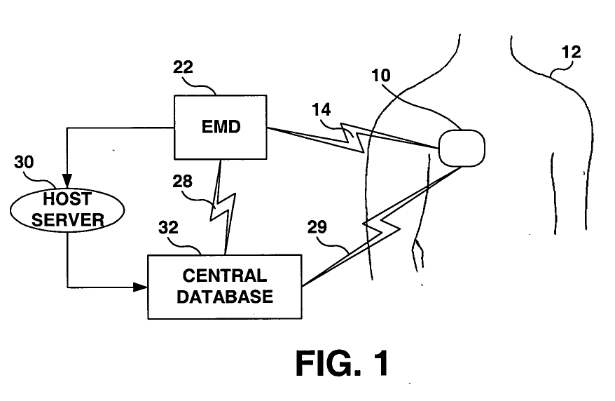

FIG. 1 illustrates a system for transmission of patient and medical device data to a centralized database for remote monitoring of the patient. The system provides continuous or periodic monitoring of patients either in a hospital setting or in a home environment. In this example, the medical device is shown as an implantable medical device (IMD) 10, implanted in a patient 12.

IMD 10 is provided with an antenna and associated circuitry, as will be described below, for establishing a bidirectional communication link 14 with an external medical device (EMD) 22, which may be embodied as a programmer or home monitor. Data acquired by IMD 10 can be transferred to the EMD 22 through communication link 14. EMD 22 is adapted to communicate with a central database 32 to allow transfer of data received from IMD 10 to the central database 32. EMD 22 transfers data via a communication link 28, which may be established via the Internet, a local area network, a wide area network, a telecommunications network or other appropriate communications network and may be a wireless communication link. EMD 22 may alternatively transfer data to a central database at a clinic via a networked host server 30. Alternatively, IMD 10 may be provided with a wireless communication module for transferring data directly to central database 32 via a wireless communication link 29. Other examples of communication schemes that may be used in remote monitoring systems are generally disclosed in U.S. Pat. No. 6,599,250 issued to Webb et al., U.S. Pat. No. 6,442,433 issued to Linberg, and U.S. Pat. No. 6,574,511 issued to Lee, U.S. Pat. No. 6,480,745 issued to Nelson et al., U.S. Pat. No. 6,418,346 issued to Nelson et al., and U.S. Pat. No. 6,250,309 issued to Krichen et al., all of which patents are incorporated herein by reference in their entirety.

Implementation of the present invention is not limited to remote monitoring of implantable medical device systems as shown in FIG. 1. In other embodiments, the remote medical device associated with a patient and from which data is retrieved for storage in a central database may be an external medical device deployed for bedside or ambulatory monitoring or therapy delivery. The external medical device would be adapted for communication with the central database as generally shown in FIG. 1.

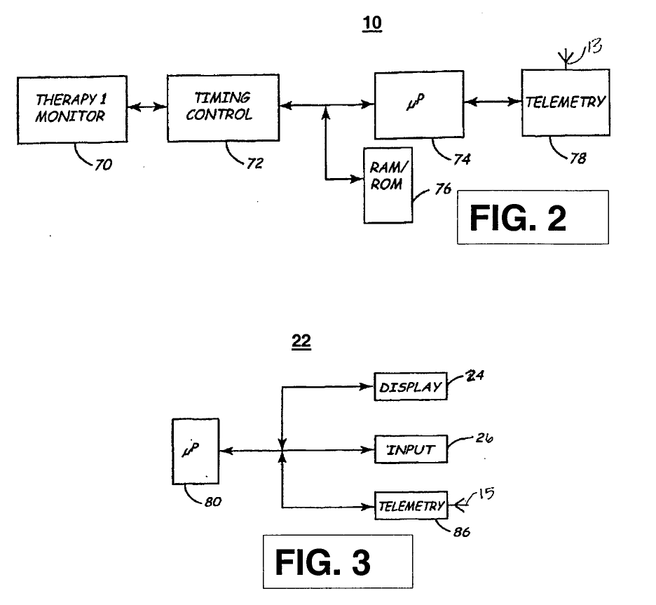

FIG. 2 illustrates typical components of IMD 10 shown in FIG. 1. Major operative structures common to IMD 10 are represented in a generic format. IMD 10 may be embodied as a cardiac pacing device, an ICD, a cardiac monitoring device, a drug delivery device, a neuromuscular stimulator, or other implantable monitoring or therapy delivery device. IMD 10 typically contains timing and control circuitry 72 and an operating system that may employ microprocessor 74 or a digital state machine for timing sensing and therapy delivery functions in accordance with a programmed operating mode. IMD 10 contains therapy/monitor 70 which may include sense amplifiers for detecting physiological signals such as cardiac electrical signals, heart wall motion signals, blood pressure signals, blood chemistry signals, respiration signals, patient activity signals or other physiologic signals for monitoring purposes or for sensing the need for delivering a therapy under control of the operating system.

The operating system includes memory registers or RAM/ROM 76 for storing a variety of programmed-in operating mode and parameter values that are used by the operating system. The memory registers or RAM/ROM 76 may also be used for storing data compiled from sensed physiological signals and/or relating to device operating history for telemetry out on receipt of a retrieval or interrogation instruction. All of these functions and operations are known in the art, and many are generally employed to store operating commands and data for controlling device operation and for later retrieval to diagnose device function or patient condition.

Programming commands or data are transmitted between IMD 10 RF telemetry antenna 13 and an external RF telemetry antenna associated with an EMD. RF telemetry antenna 13 is coupled to a telemetry transceiver 78 including a telemetry transmitter and telemetry receiver. The telemetry transmitter and telemetry receiver in IMD 10 are coupled to control circuitry and registers operated under the control of microcomputer 74.

FIG. 3 is a simplified block diagram of major functional components typically included in an EMD, such as EMD 22 shown in FIG. 1. The external RF telemetry antenna 15 on EMD 22 is coupled to a telemetry transceiver 86, which includes an antenna driver circuit board having a telemetry transmitter and telemetry receiver. The telemetry transmitter and telemetry receiver are coupled to control circuitry and registers operated under the control of microcomputer 80. Data acquired by IMD 10 for transfer to a central database is transmitted to EMD 22 via a communication link established between IMD antenna 13 and EMD antenna 15.

EMD 22 may be a personal computer type, microprocessor-based device incorporating a central processing unit 80, which may be a microprocessor. A system bus interconnects CPU 80 with a hard disk drive, storing operational programs and data, and with a graphics circuit and an interface controller module. A floppy disk drive or a CD ROM drive may also be coupled to the bus and is accessible via a disk insertion slot within the housing of EMD 22. EMD 22 may include solid-state memory for long-term storage of data. EMD 22 may also include an interface module for coupling peripheral or accessory devices or instruments to EMD 22. For example, EMD 22 may be provided with a strip chart printer, external data storage medium, ECG electrodes or other accessory devices. EMD 22 may be embodied or include features as generally disclosed in U.S. Pat. No. 5,345,362 issued to Winkler, which is incorporated by reference herein in its entirety.

In order for the clinician, patient, or other caregiver or authorized operator to interact with the EMD 22, a keyboard or other user interface input 26 coupled to CPU 80 is optionally provided. Alternatively, or in addition, communication may be conducted through graphics display screen 24 of the well-known “touch sensitive” type controlled by a graphics circuit. A user of EMD 22 may interact therewith through the use of a stylus, also coupled to a graphics circuit, which is used to point to various locations on screen or display 24 which display menu choices for selection by the user or an alphanumeric keyboard for entering text or numbers and other symbols. Various touch-screen assemblies are known and commercially available. Display 24 and/or the user interface 26 allow a user to enter command signals to initiate transmissions of downlink or uplink telemetry and to initiate and control telemetry sessions once a telemetry link with an implanted device has been established. Other types of user interaction mechanisms and electronics may be implemented such as voice recognition/response systems.

EMD 22 and IMD 10 may also interact without user intervention through long-range telemetry systems. A long range telemetry system is generally disclosed in U.S. Pat. No. 6,482,154, issued to Haubrich, et al., incorporated herein by reference in its entirety. When IMD 10 comes within communication range of EMD 22, a transfer of data between IMD 10 and EMD 22 may occur automatically, after which EMD 22 may transfer data received from IMD 10 to a central database through a wireless transmission or after establishing a cable or phone connection.

Display screen 24 is also used to display patient related data, menu choices and data entry fields used in entering the data or messages alerting a patient or user to pertinent programming or monitoring conditions. Display screen 24 also displays a variety of screens of telemetered out data or real time data. Display screen 24 may also display uplinked event signals as they are received and thereby serve as a means for enabling the user to timely review IMD operating history and status.

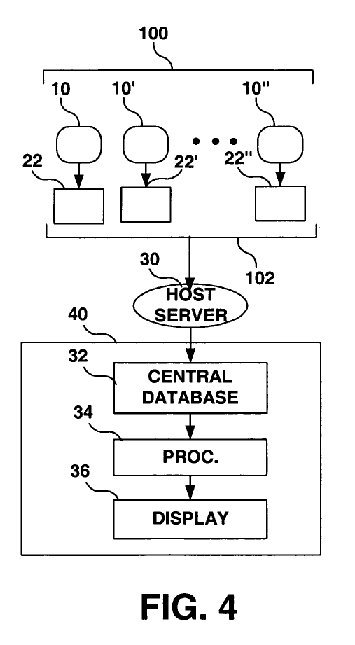

FIG. 4 is a schematic diagram illustrating a remote monitoring system configured to acquire data from multiple implantable medical devices and display summary data for multiple patients simultaneously in accordance with the present invention. A central monitoring station 40 includes central database 32, a processing unit 34, and a display 36, which is provided as a GUI in an exemplary embodiment. Central database 32 receives data from multiple, remotely located IMDs 100, via associated EMDs 102. The multiple IMDs 100 are each deployed for monitoring or treating a patient. Any given patient may be monitored or treated with one or more IMDs, each of which may provide data to the central database 32. The EMDs 102 may transfer data to central database 32 via a networked host server 30 or other appropriate communication network as described previously. The central monitoring station 40 may acquire and display data from external medical devices in addition to or rather than implantable medical devices as indicated in FIG. 4. The central monitoring station 40 may also be (in whole or in part) a system for maintaining electronic medical records.

The central monitoring station 40 includes a processing unit 34 for analyzing data received by the central database 32. Such data includes data received from IMDs 100 and may include data retrieved from electronic medical records and/or external medical devices used in monitoring or treating a particular patient. Data analysis is performed to parameterize and categorize incoming data such that summary parameter values may be displayed on display 36 in a summary format for multiple patients simultaneously as will be described in greater detail below. Algorithms for parameterizing and categorizing summary data may be implemented on processing unit 34 utilizing data stored in central database 32. Alternatively algorithms for parameterizing and categorizing summary data may be implemented at least in part as software programs on host server 30, IMD 10, or EMD 22 such that data transferred to central database 32 includes summary data that has already be parameterized and categorized. In one embodiment, detailed patient and device-related data received by host server 30 may be stored in a database provided by the host server with parameterized and categorized summary data provided to central database 32 for display on display 36. Host server 30 may be optional in that EMDs 102 or IMDs 100 may communicate directly with central database 32 via an appropriate communication link.

Display 36 is preferably an interactive display allowing a clinician or other user to view summary data for multiple patients simultaneously and select individual patients or parameters for which more detailed data review is desired. Detailed patient and device-related data may then be retrieved from central database 32 or transferred to central database 32 from host server 30 and displayed on display 36. Central database 32 and any database included on host server 30 may be embodied as a relational database providing any number of individual patient records but may also store additional files, software programs, or other data which may be accessed by the central monitoring station 40 or the medical devices 100 and 102. For example, programs or values used by processing unit 34 or the operating systems of IMDs 100, EMDs 102, or host server 30 for parameterizing and categorizing patient data in creating a summary display of multiple remotely monitored patients may be stored in central database 32 or a host server database.

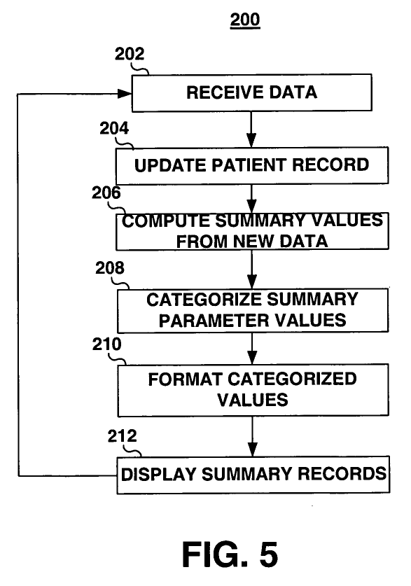

FIG. 5 is a flow chart presenting one method for obtaining and displaying patient summary data. Method 200 begins at step 202 when data is received by the central database from a remote medical device, along with any data retrieved from electronic patient records. The central database updates the corresponding patient record with the new data at step 204. Data received by the central database may include summary parameter values for display in a summary display. In other embodiments, summary parameter values are computed from data received by the central database at step 206. A number of monitored parameters to be included in a summary display are predefined. The monitored parameters available for display will in part depend on the type of device the patient has and the particular monitoring features that are enabled.

At step 208, parameter values are categorized. Two or more categories are predefined based on a clinician's interest in reviewing summary data. For example, parameter values may be categorized according to whether or not a patient is due for a remote monitoring session. The central database may also periodically check to see if a patient is overdue for a remote monitoring session and flag or highlight such patients. Parameter values may additionally or alternatively be categorized according to a change or a degree of change in a parameter value since a previous monitoring session. In another embodiment, parameter values are categorized according to whether the value warrants clinical attention, for example as a pathologically high or low value for blood pressure, heart rate or other physiological signal. As such, categorizing of parameter values may include a comparative analysis of a parameter value with predefined thresholds, ranges or other previously stored values.

Categorized parameter values are formatted at step 210 according to a predetermined formatting scheme which allows the category of displayed summary values to be distinguished. For example, different symbols, color, font, highlighting, shading, or other formatting schemes may be used to differentiate between categories. In particular, values that require updating or are of particular clinical concern may be formatted for display in a conspicuous manner to draw the attention of a clinician or other user.

Summary patient records for multiple patients are then displayed simultaneously at step 212. The summary display may be provided in a generally tabular form with each of the summary parameter values for each patient appropriately formatted according to category. A user is then able to scan the summary display, scrolling up and down through the data as necessary, to quickly recognize any patients having monitored parameters categorized as indicating a possible need for medical attention or otherwise being of clinical interest. At any time new data is received by the central database, method 200 may be repeated by returning to step 202 and proceeding to update appropriate patient records, determine and categorize new summary parameter values, and generate a formatted summary display.

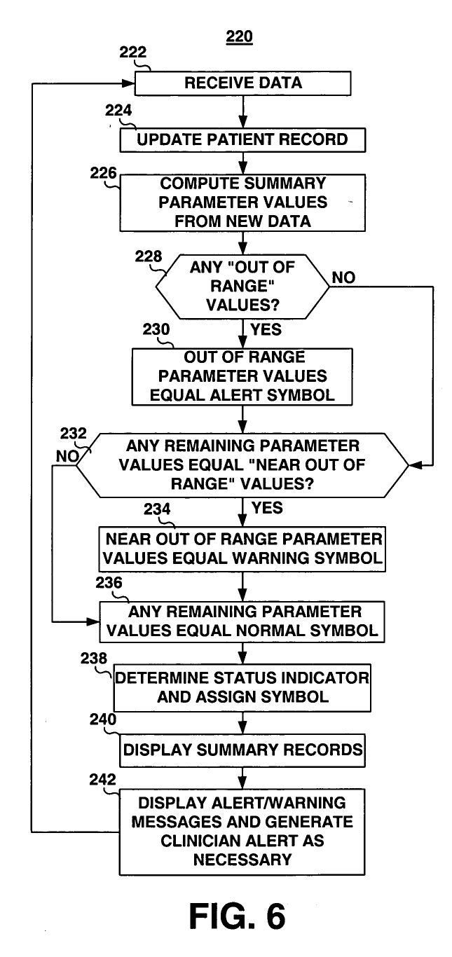

FIG. 6 is a flow chart categorizing and displaying summary data for patients being monitored remotely. When new data is received by a central database at step 222, the corresponding patient record is updated at step 224, and parameter values to be included in a summary display are computed at step 226 using the new data. The parameter values are then categorized according to predefined thresholds or ranges. In this example, parameter values are categorized according to: 1) an “alarm” category, indicating the patient may require medical attention based on the parameter value; 2) a “warning” category, indicating the patient may soon require medical attention based on the parameter value; and 3) a “normal” category, indicating the parameter value is within an acceptable range and does not indicate a need for clinical attention.

The categorizing of summary parameter values according to these exemplary categories is summarized by steps 228 through 236 shown in FIG. 6. At decision step 228, the monitored parameter values are compared to predefined thresholds or ranges to determine if any parameter values are “out of range.” An “out of range” value is a value that has crossed a predetermined threshold or fallen outside a normally accepted range for the given parameter and is an indication that the patient may require clinical attention. An “out of range” value may be arrived at through a simple threshold crossing of the parameter, a crossing of an automatically adjusting threshold, e.g. according to methods as generally disclosed in U.S. patent application Nos. P-11119.00 and P-20485.00 to Stadler et al., or using more advanced signal processing of one or more parameters and their historical changes. In some embodiments, an “out of range” value may be a parameter that has changed substantially from a previously measured value or from a baseline value established for the given patient. Definitions for “out of range” values for any summary parameter may be predetermined and stored by the central database and used by a processing unit in categorizing parameter values. A clinician may customize “out of range” definitions for any summary parameter according to individual patient need or according to the clinician's personal preferences for a particular patient group that he/she is treating. Categorizing a parameter value may include a comparative analysis of the parameter value and an established baseline for a particular patient or values set based on clinical experience. “Out of range” criteria involving multiple parameters may be predefined or set by a clinician. At step 230, any parameter values found to be “out of range” are formatted for display as an “alert” symbol which will be displayed as the parameter value in the summary display. Categorizing a parameter value in the “alert” category may trigger an alarm at the clinic, from the IMD and/or on the EMD. An alert notification may be sent to the clinic or clinician using a communication medium such as an email, a fax, a page or a phone call.

At decision step 232, the parameter values for any remaining parameters that are not categorized as “out of range” are compared to predefined thresholds or ranges to determine if any parameter values are near or approaching the “out of range” criteria. A “near out of range” value may be a value that has come within some predetermined range or percentage of a threshold defining the “out of range” boundary. Alternatively, uniquely defined thresholds or ranges may be used to define both the “out of range” and “near out of range” categories for any given parameter. At step 234, any parameter values found to be “near out of range” are assigned a “warning” symbol to be displayed as the parameter value in the summary display. A “warning” symbol indicates the patient may soon require medical attention based on the corresponding parameter value. The user may then choose to monitor that patient more frequently, adjust a therapy, schedule an office visit or take other appropriate action for either preventing the parameter from becoming “out of range” or enabling a quick response in case it does become out of range.

All remaining parameter values, which are not found to be “out of range” or “near out of range,” are assigned a “normal” symbol at step 236. These parameter values do not indicate a need for clinical attention at the present time according to the category definitions. At step 238, an overall status indicator may be determined for each patient based on the patient's parameter categories or values. An overall status indicator may be assigned an alert symbol if one or more parameter values are categorized as “out of range”; a warning symbol if one or more parameter values are categorized as “near out of range”; or a normal symbol if all parameter values are categorized as “normal.” An overall status indicator may be determined based on a subset of summary parameter values which may be considered to be greater clinical importance in determining a patient's status than other parameter values. As such, a patient may have some parameter values categorized as “out of range” but if other parameter values are categorized as “normal” and are considered to be of greater clinical importance, an overall status indicator may still be assigned a normal or warning symbol for display in a summary display. Likewise, related parameters such as those related to hemodynamic function, device operation, or heart failure status may be grouped and summarized.

Additionally or alternatively, a physiologic status indicator may be determined at step 238 based on one or more physiological parameters. Physiological signals monitored by the IMD as well as measures entered into a patient's electronic record or monitored by external devices may be used in determining a physiologic status indicator. Parameters used in determining a physiologic status indicator may include, for example, weight change, reported symptoms, blood pressure measurements or other monitored hemodynamic measures, blood chemistry parameters such as oxygen levels, glucose levels and electrolytes, and arrhythmia history. A physiologic status indicator does not consider device-related parameters whereas an overall status indicator may consider both physiologic and device-related parameters. As will be described below, a separate device status indicator may be determined based on one or more device-related parameters to indicate device performance.

At step 240, the summary records for multiple remotely monitored patients are displayed including, in each patient record, the assigned symbols for the categorized parameter values, and optionally the overall status indicator. The category symbols may be selected as icons or color-coded symbols and may include other formatting which allows a clinician or other user to easily distinguish between parameter value categories. For example, an “alarm” symbol may be displayed as a red symbol, a “warning” symbol may be displayed as a yellow symbol, and a “normal” symbol may be displayed as a green symbol. An index or legend of category symbols or formatting may be included in the summary display. Any time new data is received by the central database, the summary display may be updated by returning to step 222 and repeating method 220.

Upon updating a summary display with current categorized parameter values or symbols, a clinician alert may be generated at step 242. Alarm or warning messages may be displayed based on summary parameter values. In addition, medical personnel may be alerted via a communication medium such as electronic mail, electronic page, fax, or telephone call. Thus, the central monitoring station is coupled to an appropriate communication network to allow alert or warning messages generated according to summary parameter values to be transferred to a network destination, telephone or pager number.

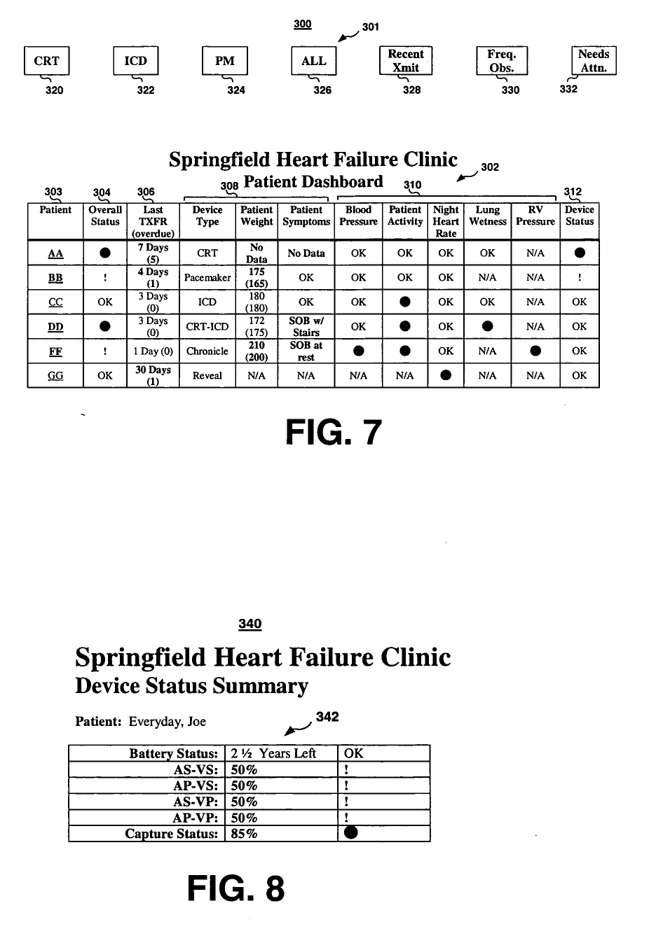

FIG. 7 illustrates one embodiment of an interactive display of summary data for multiple patients being monitored remotely. Interactive display 300 provides a tabular listing of summary parameter values for multiple patients being monitored remotely by a clinic. The display 300 includes patient grouping buttons 301 and a summary data table 302 corresponding to a selected patient group. Patient records simultaneously displayed in summary table 302 may be selected according to various grouping criteria. A user selects a particular patient group for display in summary table 302 by selecting one of the grouping buttons 301. Fields, cells, or buttons included in display 300 may be selected by a user using a keyboard, pointing device, voice recognition system or other user interface technology.

In the example shown, patient groups are selectable using buttons 320 through 332 according to the type of medical device, time since the most recent remote monitoring session in which data was transferred from the medical device to the central database, the frequency of required clinical attention, or an indication of required clinical attention. As such, a cardiac resynchronization therapy (CRT) button 320, an implantable cardioverter defibrillator (ICD) button 322, and a pacemaker (PM) button 324 are provided for selecting a group of patients having these respective medical devices. All patients being monitored by the clinic or a particular clinician may be selected using the ALL button 326.

Selection of the “Recent Xmit” button 328 allows a clinician to view summary data for patients that have had data added to their records within a recent interval of time or since the last summary display 300 was viewed. In some embodiments, a time interval defining recent transmissions may be selectable by the clinician based on the frequency he/she monitors the summary display. Recent transmissions can include data for patients that has not been viewed previously by the clinician.

A patient group may defined according to the frequency of required medical attention. Patients requiring frequent medical care or observation may be selected using a “Freq. Observation” button 330. Patients to be included a “frequent observation” group may be assigned to the group by a clinician as those patients having frequent symptoms, complications, hospitalizations or other clinical needs. These are patients the clinician would like to closely monitor on a more frequent basis than the typical patient.

A “needs attention” button 322 is provided for selecting a patient group that includes patients having one or more summary parameters categorized as “out of range” or “near out of range” or otherwise are determined to have an indication that warrants medical attention. By selecting the “needs attn.” button 332, a group of patients having one or more parameters indicating a potential need for medical attention is displayed.

An exemplary formatting scheme for summary table 302 is shown in FIG. 7. Summary table 302 includes a number of columns assigned to summary parameters and a number of rows in which each patient record is displayed. Summary parameter values are displayed in corresponding cells defined by the respective columns and rows. The order in which patients in a selected patient group are listed in table 302 may be determined by sorting the summary data according to any of the column parameters. In the example shown, patients are listed alphabetically according to patient name shown in the first column 303.

The remaining columns provide summary parameter values for each respective patient. An overall status indicator is shown in the second column 304. The overall status indicator may be displayed as a coded icon or color-coded symbol indicating if the patient requires medical attention based on the categorized parameters values as described previously. In the formatting scheme shown in FIG. 7, an exclamation point is used as an alert symbol indicating a need for clinical attention. A solid circle is used as a warning symbol, and an “OK” is used as a normal symbol. In other embodiments, color coded symbols may be used to distinguish parameter value categories. For example, a red symbol may be used as an alert symbol; a yellow symbol as a warning symbol; and a green symbol as a normal symbol. Display 300 may include a legend of category symbols or formatting.

A user may be able to select the “Overall status” column 304 to cause the displayed data to be sorted according to the overall status indicator. In this way, patients having an alarm symbol as the overall status value to indicate that the patient needs clinical attention will be listed first followed by patients having a warning symbol and finally patients having a normal symbol. In this way, the clinician is able to view a list of patients and quickly identify those having an indication for clinical attention.

In column 306, the number of days since the last remote monitoring session in which data was transmitted from the medical device to the central database is listed. The number of days shown may be color-coded or formatted in a way that indicates if a transmission is overdue. A user may be able to select the “Last TXFR” column 306 for data sorting such that patients that are overdue for a data transfer from their medical device to the central database are listed above patients having up-to-date data transfers. In this way, scheduling or follow-up of patients overdue for a remote monitoring session is facilitated.

A number of columns 308 may be included for displaying patient information that may be entered into a patient record by a clinician but is not necessarily collected by a medical device. For example, columns for entering the device type, the patient weight, complaints of symptoms, or other patient history may be included. Data may be retrieved from an electronic medical records system or from other medical devices. In some embodiments, a patient may be able to enter information such as their current weight, current systemic blood pressure, current symptoms (or lack of symptoms) using a home monitor to allow the patient to include such information with other data collected and transmitted by the medical device to the central database. Any of these columns 308 may be selected for sorting the order in which patient records are displayed.

In some embodiments hierarchal sorting of patient records may be performed. In hierarchal sorting, patient records are listed in an order determined by sorting first by a primary parameter, second by a secondary parameter, and so on. In one example, the patient group may be listed in an order determined by sorting first according to an overall status indicator and second according to an entry in the patient symptoms column. Patients having an overall status indicating a need for clinical attention and having a complaint of symptoms entered would be listed first in the table followed by patients having an overall status indicating a need for clinical attention but no entry of symptoms, followed by patients having symptoms but a normal overall status indicator and lastly patients having no symptoms recorded and a normal overall status indicator.

Other parameters listed in summary table 302 can include any physiological parameters that are monitored by the medical device. In the example shown in FIG. 7, parameters relating to blood pressure, patient activity, night time heart rate, lung wetness and right ventricular pressure are listed in respective columns 310. For a given patient group, it is possible that not all parameters displayed in summary table 302 will be available for all patients due to different types of medical devices or device features enabled. In some cells corresponding to a respective parameter and patient “N/A” or other similar meaning notation may be displayed to indicate that the parameter value is not available from the data stored in the central database.

In the exemplary embodiment shown in FIG. 7, each parameter is categorized for each listed patient so that a category symbol can be displayed in each respective cell in columns 310. Categorized parameter values are thus displayed as category symbols. In alternative embodiments, an actual measurement value for a given parameter may be displayed in a respective cell. An actual measurement value may be derived from physiological signals sensed by the medical device. Such derivation may be performed by the medical device with the resultant value transferred to the central database. Alternatively, parameter values may be derived by processing units included in the clinical monitoring station or a host server using raw or processed physiological signals received from the medical device. Actual measurement values displayed may represent a long or short term average, a change from a baseline value, or any other statistical or derived value representative of the monitored parameter.

A derived value may be categorized as described previously and formatted for display in a conspicuous manner to indicate the category. For example, a derived value that falls into an “out of range” category may be highlighted or displayed in particular color, such as red, to draw attention to the value. Values falling into a “near out of range” category may be displayed or highlighted in yellow, and values falling into a normal category could be displayed in normal black or green text.

The clinician's data review burden is substantially lessened by formatting categorized parameter symbols or measurements in a manner that allows the clinician to quickly identify values of clinical concern. Multiple data parameters may be condensed into indices or summary values to further reduce the data that must be reviewed by a clinician. The entries in columns 310 could be summarized or indexed data, based on one or more parameters recorded by the IMD or an external medical device. For instance, the blood pressure indicator may be a single, actual blood pressure measurement (internal or external), but may alternatively be a computed blood pressure index or trend determined from multiple external and/or internal blood pressure measurements. In one example, a blood pressure index may be computed from internally measured RV pulse pressure, RV dP/dt, estimated pulmonary artery pressure, or other RV pressure related measurements. Selecting a cell in summary table 302 containing a summary or indexed parameter value or symbol will cause detailed data used in generating the value or symbol to be displayed.

Summary table 302 further includes a device status column 312 for listing an overall medical device status indicator. A device status indicator may be categorized according to the status of one or more medical device-related parameters. Device-related parameters that may be used in determining an overall device status indicator can relate to expected battery life, therapies delivered, lead impedance, capture detection, threshold tests, and results of any other self-diagnostic tests that the device may be enabled to perform. If one or more device-related parameters indicate a potential need for clinical attention, the device status indicator may be formatted accordingly for display. In alternative embodiments, other device-related parameters may be displayed in addition or alternatively to an overall device status indicator.

In general, a clinician needs to know whether the device operation is within acceptable bounds or not. Normal or acceptable device operation can be succinctly indicated by a symbol or icon, without requiring the clinician to review an extensive history of device performance or results of self-diagnostic tests. As such, analysis of device-related parameters may be performed by the medical device or by a processing unit located at a host server or the central monitoring station for determining and categorizing an overall device status indicator. The appropriate category symbol is then displayed in the device status column 312. Acceptable bounds or limits for device operation may be predefined and may be selectable by a clinician and may be made patient-specific. If any device status indicator is categorized as indicating a need for clinical attention, the clinician may select the respective cell to cause a more detail listing of device-related parameters to be displayed.

In an exemplary embodiment, a clinician or user may select any cell within summary table 302 in order to view additional data relating to the categorized parameter value. For example, data used in categorizing the parameter value by a processing unit may be provided in a more detailed display for view by a clinician.

FIG. 8 is an illustration of a user-interactive display listing data relating to a device status indicator. A clinician may select a cell displaying a device status indicator for a given patient in summary table 302 (FIG. 7) in order to review device status data in greater detail. In one embodiment, selecting a device status indicator cell in a summary table 302, causes device status summary screen 340 shown in FIG. 8 to be displayed.

The device status summary screen 340 lists the respective patient's name and provides a listing 342 of device status parameters and their respective values. Device-related parameters displayed will depend on the device type. In the example shown, the patient is a pacemaker patient and the device status parameters listed include battery status; the percentage of cardiac cycles in which both atrial and ventricular chambers were sensed (AS-VS); the percentage of cardiac cycles in which the atrial chamber was paced and the ventricular chamber was sensed (AP-VS); the percentage of cardiac cycles in which the atrial chamber was sensed and the ventricular chamber was paced (AS-VP); the percentage of cardiac cycles in which both chambers were paced (AP-VP); and the percentage of pace events for which capture was verified. The capture success rate may further be expanded to display the frequency of capture at each pacing site. For example, the capture frequency in the right ventricle, left ventricle, right atrium and left atrium may be distinctly listed. Other device status parameters that could be displayed include lead impedance measurements or lead status, threshold measurements, operation modes, number of arrhythmia episodes or other physiological events detected, and therapies delivered.

Each device status parameter may be categorized and formatted for display accordingly. In one embodiment, the category may be indicated in a third column as shown in FIG. 8 using category symbols or formatting consistent with the scheme used in the summary table 302. In alternative embodiments, the parameter names and values may be displayed in color-coded text to indicate the category.

As noted above, any cell may be selectable by a user for causing a more detailed display of data related to the respective parameter value. Such detailed displays may include histograms, strip chart like recordings, long term parameter trends, data plots or other graphical displays and/or tabular lists of data. Data points or values of particular interest may be highlighted using symbols or other formatting that may be consistent with the category formatting scheme used in summary table 302.

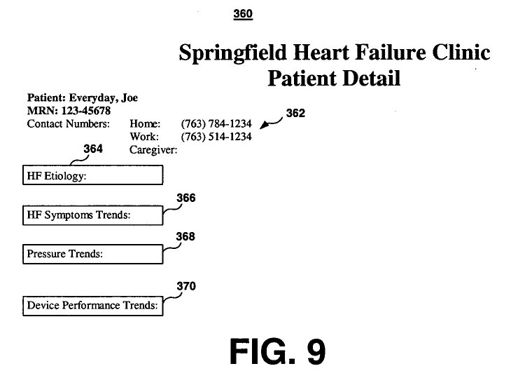

FIG. 9 illustrates an interactive display listing detailed patient data. A clinician may select a patient's record or name from summary table 302 (FIG. 7) or similar button on any other sub-pages arrived at by clicking on the patient name (FIG. 8, patient name) to cause a display of additional patient information for the selected patient. The patient's name and a medical records number may be shown at the top of the detailed patient data display 360 followed by patient contact information 362. Patient contact information may be provided to allow a clinician to quickly attempt to contact the patient for scheduling a clinical visit, a remote monitoring session or otherwise advise the patient. Additional patient and device-related data may also be listed including graphs or tables, which may in turn may be selected in order to view greater detail.

In the example shown in FIG. 9, various patient history buttons may be selected to cause the interactive display to provide a view of more detailed data. For example, a heart failure (HF) etiology button 364 may be selected to review clinical notes stored in the central database or other electronic patient medical record regarding the patient's diagnosis and disease progression. A HF symptoms trends button 366 may be selected to review a chronological listing or other tabular or graphical display of clinical data or clinician or patient notes regarding HF symptoms. A pressure trends button 368 may be selected to review a graphical or tabular display of blood pressure measurements or other hemodynamic measurements or changes over time. A device performance trends button 370 may be selected to review a graphical or tabular display of changes in device operation occurring over time, such as the frequency of delivered therapies, types of therapy, or other device performance measurements. In some embodiments, more detailed views of data trends may be correspond to views provided in a quick look summary for patient management systems as generally disclosed in U.S. Pat. No. 6,599,250 issued to Webb et al., previously incorporated herein. Detailed views of patient or device-related data may correspond to views provided by known GUIs used with medical devices, for example as generally disclosed in U.S. Pat. No. 6,583,796 issued to Jamar et al., hereby incorporated herein by reference in its entirety.

Thus a remote patient monitoring system has been described which includes an interactive display of summary data for multiple patients simultaneously. Such summary data is categorized and displayed in a manner that allows a clinician or user to quickly ascertain summary data parameters that are of clinical importance. The interactive display further allows the clinician or other user to delve down into the summary data in order to view more detailed data by selecting any patient name or data cell to cause more detailed data relating to the respective patient or categorized parameter value to be displayed. The system provides a clinician with a time-saving tool for quickly recognizing patients that may require clinical attention without the burden of lengthy data review for all patients. Furthermore, clinicians and patients may be saved the time and inconvenience of routine periodic office visits that are unwarranted when the patient is feeling well and device operation is appropriate.

Various aspects of the present invention have been described herein according to detailed, illustrated embodiments. However it is recognized that numerous variations may be conceived for categorizing, arranging, formatting, and displaying summary parameter data for multiple patients simultaneously, from which a clinician or other user can quickly recognize which patients within a selected group may need clinical attention or other follow-up. Likewise, all possible parameters of interest that could be collected by an IMD or EMD may not have been mentioned, but could be included in this general invention. The various embodiments described herein should therefore be considered exemplary, not limiting, with regard to the following claims.

What is claimed is:

1. A system for remote patient monitoring, comprising:
a central database;
a communication network for transferring data to the central database from a plurality of remote medical devices deployable for monitoring or treating a plurality of patients;
a processor for analyzing the data received by the central database for each of the plurality of patients in order to parameterize and categorize the data according to a plurality of categories; and
a graphical user interface including a summary table of patient records for displaying categorized data parameters for a number of the plurality of patients simultaneously.

2. The system of claim 1, wherein the graphical user interface displays categorized data parameters according to a formatting scheme designed to visually distinguish between parameter categories.

3. The system of claim 1, wherein the plurality of categories includes an alarm category to which a parameter value is assigned when the patient is highly likely to need clinical attention.

4. The system of claim 3 further including a warning category to which a parameter value is assigned when the value is approaching the alarm category.

5. The system of claim 1, wherein the graphical user interface further allows a user to select a cell within a displayed summary table to cause the graphical user interface to display a view of detailed data stored by the central database relating to the selected cell.

6. The system of claim 5, wherein the graphical user interface further allows a user to select a field in the display of detailed data to cause the graphical user interface to display a view of relatively more detailed data.

7. The system of claim 1, wherein the graphical user interface further includes a plurality of patient grouping buttons corresponding to predefined patient groups and wherein a patient grouping button is selectable by a user to cause the graphical user interface to display a summary table of patient records for patients falling into the selected patient group.

8. A method for remote patient monitoring, comprising:
transferring data from a plurality of medical devices to a central database;
processing transferred data to generate a set of summary parameter values;
categorizing the summary parameter values according to a plurality of predefined categories;
formatting categorized summary parameter values to allow the category of a parameter value to be visually distinguishable; and
displaying formatted, categorized summary parameter values for a plurality of patients simultaneously in a summary table.

9. The method according to claim 8 wherein one of the plurality of predefined categories is a category indicating a need for clinical attention and parameter values categorized as indicating a need for clinical attention are formatted in a conspicuous manner.

10. The method according to claim 9 wherein parameter values categorized as indicating a need for clinical attention are formatted in red.

11. The method according to claim 8 wherein formatting of categorized parameter values includes displaying the parameter value as a symbol corresponding to the assigned category.

12. The method according to claim 8 wherein processing transferred data further includes determining an overall patient status indicator.

13. The method according to claim 12 wherein the overall patient status indicator corresponds to the category of one or more summary parameter values.

14. The method according to claim 12 wherein the overall patient status indicator is formatted for display according to the category of one or more summary parameter values.

15. The method according to claim 8 wherein processing transferred data further includes determining a device status indicator.

16. The method according to claim 8 wherein processing transferred data further includes determining a physiologic status indicator.

17. The method according to claim 8 further including displaying patients according to predefined patient groups.

18. The method according to claim 8 further including sorting patient records for ordering the display of the patient records in a summary table according to categorized summary parameter values.

19. The method according to claim 8 further including displaying a warning or alert message based on summary parameter values.

20. The method according to claim 8 further including transferring a warning or alert message according to summary parameter values to a communication network destination.

21. A system for remote patient monitoring, comprising:
means for storing medical data;
means for transferring medical data from a plurality of medical devices to medical data storing means;
means for determining summary parameter values for each of a plurality of patients from data stored in medical data storing means;
means for categorizing the summary parameter values according to a plurality of predefined categories;
means for formatting the summary parameter values according to category; and
means for displaying formatted, categorized summary parameter values for a plurality of patients simultaneously.

22. A computer-readable medium for storing a set of instructions which when implemented causes a remote patient monitoring system to:
categorize parameter values determined from medical data received by a central database from a remote medical device;
format the parameter values according to category; and
display a summary table of formatted, categorized parameter values for a plurality of patients simultaneously wherein the formatting of the categories of displayed parameter values are visually distinguishable.
